(19) United States
(12) Patent Application Publication
(10) Pub. No.: US 2006/0287339 A1
(43) Pub. Date: Dec. 21, 2006

(54) Aza- and polyazanthranyl amides and their use as medicaments

(76) Inventors: Dieter Seidelmann, Berlin (DE); Martin Kruger, Berlin (DE); Orlin Petrov, Berlin (DE); Andreas Huth, Berlin (DE); Karl-Heinz Thierauch, Berlin (DE); Andreas Menrad, Oranienburg (DE); Martin Haberey, Berlin (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/510,416

(22) Filed: Aug. 25, 2006

Related U.S. Application Data
(63) Continuation of application No. 10/275,584, filed on May 9, 2003, now abandoned.

(30) Foreign Application Priority Data
May 9, 2000 (DE) 10023492.5

(51) Int. Cl. A61K 31/497; A61K 31/506; C07D 403/02

(52) U.S. Cl. 514/255.05; 544/405; 514/275; 544/331

(57) ABSTRACT

Aza- and polyazanthranyl amides and their use as medicaments in the treatment of diseases caused by persistent angiogenesis are described, as well as the intermediates used in the preparation of the aza- and polyazanthranyl amides.

The invention relates to substituted aza- and polyazanthranyl amides and their use as medicaments in the treatment of diseases caused by persistent angiogenesis, as well as the intermediates used in the preparation of the aza- and polyazanthranyl amides.

Persistent angiogenesis may be the cause of various diseases such as psoriasis, arthritis, such as rheumatoid arthritis, haemangioma, angiofibroma, eye diseases such as diabetic retinopathy, neovascular glaucoma, kidney diseases such as glomerulonephritis, diabetic nephropathy, malignant nephrosclerosis, thrombic microangiopathic syndrome, transplantation rejections and glomerulopathy, fibrotic diseases such as cirrhosis of the liver, mesangial cell proliferation diseases, artheriosclerosis and injuries to nerve tissue, or may lead to a worsening of these diseases.

The direct or indirect inhibition of the VEGF receptor can be used to treat such diseases and other VEGF-induced pathological angiogenesis and vascular permeable conditions, such as tumour vascularisation. For example, it is known that the growth of tumours can be inhibited by soluble receptors and antibodies to VEGF.

Persistent angiogenesis is induced by the VEGF through its receptor. So that VEGF can display this activity, it is necessary for VEGF to bind to the receptor and for tyrosine phosphorylation to develop.

It has now been found that compounds of general formula I 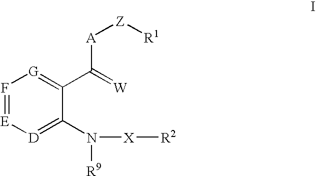
in which
A is the group═NR⁷,
W is oxygen, sulfur, two hydrogen atoms or the group ═NR⁸,
Z is a bond, the group═NR¹⁰ or═N—, branched or unbranched C₁₋₂-alkyl or the group

Alkyl is understood to be in each case a straight-chain or branched alkyl radical, such as methyl, ethyl, propyl, isopropyl, butyl, isobutyl, sec.-butyl, pentyl, isopentyl or hexyl, whereby C₁₋₄-alkyl radicals are preferred. 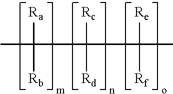
m, n and o are 0-3,
Rₐ, R_{b}, R_{c}, R_{d}, Rₑ, R_{f} independently of one another, are hydrogen, fluorine, C₁₋₄-alkyl or the group═NR¹⁰ and/or Rₐ and/or R_{b} with R_{c} and/or R_{d} or R_{c} with Rₑ and/or R_{f} may form a bond, or up to two of radicals Rₐ-R_{f} may close a bridge to R¹ or to R⁷ each with up to three carbon atoms,
R¹ is branched or unbranched C₁₋₁₂-alkyl or C₂₋₁₂-alkenyl which is optionally substituted once or many times by halogen, hydroxy, C₁₋₆-alkyloxy, aralkyloxy, C₁₋₆-alkyl and/or NR¹¹R¹²; or C₃₋₁₀-cycloalkyl or C₃₋₁₀-cycloalkenyl which is optionally substituted once or many times by halogen, hydroxy, C₁₋₆-alkyloxy, C₁₋₆-alkyl and/or NR₁₁R¹²; or aryl or hetaryl which is optionally substituted once or many times by halogen, hydroxy, C₁₋₆-alkyloxy, aralkyloxy, C₁₋₆-alkyl and/or by C₁₋₆-alkyl Which is substituted once or many times by halogen,
X is C₁₋₆-alkyl;
R² signifies monocyclic aryl, bicyclic aryl or heteroaryl, which is unsubstituted or optionally substituted once or many times by halogen, C₁₋₆-alkyl, C₁₋₆-alkoxy and/or hydroxy and
D signifies N or C—R³,
E signifies N or C—R⁴,
F signifies N or C—R⁵, and
G signifies N or C—R⁶, whereby
R³, R⁴, R⁵ and R⁶ are hydrogen, halogen; or C₁₋₆-alkoxy, C₁₋₆-alkyl, C₁₋₆-carboxyalkyl either unsubstituted or optionally substituted once or many times by halogen,
R⁷ is hydrogen or C₁₋₆-alkyl or with Rₐ-R_{f} forms a bridge of Z or to R¹ with up to 3 ring members,
R⁸, R⁹ and R¹⁰ are hydrogen or C₁₋₆-alkyl and
R₁₁ and R¹² are hydrogen, C₁₋₆-alkyl, or form a ring which may contain a further hetero atom,
   whereby if D is N, then E, F and G may not simultaneously be C—R⁴, C—R⁵ or C—R⁶ or D, E, F and G may not simultaneously be C—R³, C—R⁴, C—R⁵ or C—R⁶, as well as the isomers and salts thereof, stop tyrosine phosphorylation or persistent angiogenesis and thus prevent the growth and spread of tumours.

If R⁷ forms a bridge to R¹, heterocycles are produced, to which R¹ is condensed. The following may be mentioned by way of example:

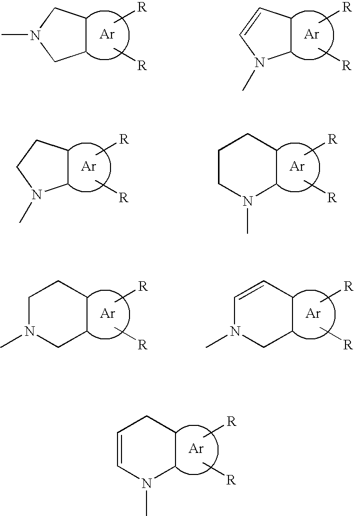

If Rₐ, R_{b}, R_{c}, R_{d}, Rₑ, R_{f} independently of one another are hydrogen or C₁₋₄-alkyl, then Z forms an alkyl chain.

If Rₐ and/or R_{b} with R_{c} and/or R_{d} or R_{c} and/or R_{d} with Rₑ and/or R_{f} form a bond, then Z is an alkenyl or alkinyl chain.

If Rₐ-R_{f} form a bridge by themselves, then Z is a cycloalkyl or cycloalkenyl group.

If up to two of radicals Rₐ-R_{f} form a bridge with up to 3 carbon atoms to R¹, then Z together with R¹ is a benzo- or hetaryl-condensed (Ar) cycloalkyl. The following may be mentioned by way of example:

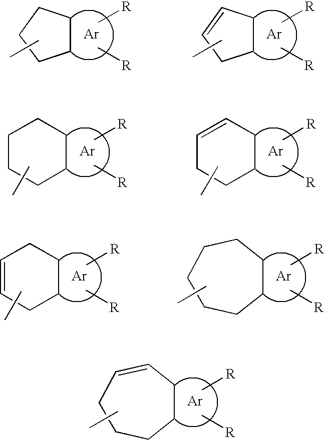

If one of radicals Rₐ-R_{f} closes a bridge to R⁷, then a nitrogen heterocycle is formed, which can be separated from R¹ by a group. The following may be mentioned by way of example:

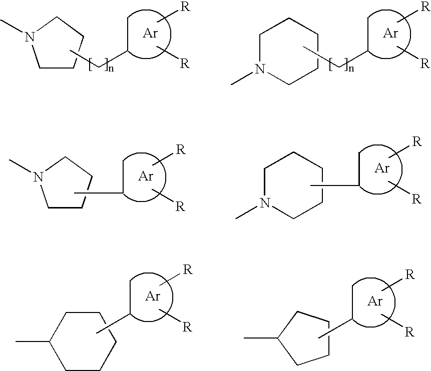

By alkyl is understood in each case a straight-chain or branched alkyl radical, such as methyl, ethyl, propyl, isopropyl, butyl, isobutyl, sec. butyl, tert.-butyl, pentyl, isopentyl or hexyl, heptyl, octyl, nonyl, decyl, undecyl, dodecyl.

By cycloalkyl is understood monocyclic alkyl rings, such as cyclopropyl, cyclobutyl, cyclopentyl, cyclohexyl oder cycloheptyl, cyclooctyl, cyclononyl or cyclodecyl, and also bicyclic rings or tricyclic rings, for example adamantanyl.

By cycloalkenyl is understood in each case cyclobutenyl, cyclopentenyl, cyclohexenyl, cycloheptenyl, cyclooctenyl, cyclononenyl or cyclodecenyl, whereby linking may take place at the double bond and also at the single bonds.

Halogen is understood to be in each case fluorine, chlorine, bromine or iodine.

The alkenyl substituents are respectively straight-chained or branched and contain 2-6, preferably 2-4 carbon atoms. The following radicals may be mentioned by way of example: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, but-3-en-1-yl, allyl.

The aryl radical respectively has 6-12 carbon atoms, for example naphthyl, biphenyl and in particular phenyl.

The heteroaryl radical may be respectively benzocondensed. Examples of 5-ring heteroaromatics are: thiophene, furan, oxazole, thiazole, imidazole and benzo derivatives, and examples of 6-ring heteroaromatics are pyridine, pyrimidine, triazine, quinoline, isoquinoline and benzo derivatives.

The aryl and heteroaryl radical may respectively be substituted 1, 2 or 3 times by the same or different substituents, from hydroxy, halogen, C₁₋₄-alkoxy, C₁₋₄-alkyl, or C₁₋₄-alkyl substituted once or more by halogen.

If an acidic function is contained therein, suitable salts are the physiologically acceptable salts of organic and inorganic bases, for example the readily soluble alkali and alkaline earth salts, as well as N-methyl-glucamine, dimethyl glucamine, ethyl glucamine, lysine, 1,6-hexadiamine, ethanolamine, glucosamine, sarcosine, serinol, tri-hydroxy-methyl-amino-methane, amino-propanediol, Sovak base, 1-amino-2,3,4-butanetriol.

If a basic function is contained therein, suitable salts are the physiologically acceptable salts of organic and inorganic acids, such as hydrochloric acid, sulphuric acid, phosphoric acid, citric acid, tartaric acid, fumaric acid, etc., as well as the isomers and salts thereof.

Of particular interest are those compounds of the general formula I, in which
A is the group═NR⁷,
W is oxygen, sulphur, two hydrogen atoms or the group═NR⁸,
Z is a bond,
R¹ is branched or unbranched C₁₋₁₂-alkyl or C₂₋₁₂-alkenyl which is optionally substituted once or many times by halogen, hydroxy, C₁₋₆-alkyloxy, aralkyloxy, C₁₋₆-alkyl and/or NR¹¹R¹²; or C₃₋₁₀-cycloalkyl or C₃₋₁₀-cycloalkenyl which is optionally substituted once or many times by halogen, hydroxy, C₁₋₆-alkyloxy, C₁₋₆-alkyl and/or NR¹¹R¹²; or aryl or hetaryl which is optionally substituted once or many times by halogen, hydroxy, C₁₋₆-alkyloxy, aralkyloxy, C₁₋₆-alkyl and/or by C₁₋₆-alkyl which is substituted once or many times by halogen,
X is C₁₋₆-alkyl;
R² signifies monocyclic aryl, bicyclic aryl or heteroaryl, which is unsubstituted or optionally substituted once or many times by halogen, C₁₋₆-alkyl, C₁₋₆-alkoxy and/or hydroxy and
D signifies N or C—R³,
E signifies N or C—R⁴,
F signifies N or C—R⁵, and
G signifies N or C—R⁶, whereby
R³, R⁴, R⁵ and R⁶ are hydrogen, halogen; or C₁₋₆-alkoxy, C₁₋₆-alkyl, C₁₋₆-carboxyalkyl either unsubstituted or optionally substituted once or many times by halogen,
R⁷ is hydrogen or C₁₋₆-alkyl,
R⁸ and R⁹ and are hydrogen or C₁₋₆-alkyl and
R¹¹ and R¹² are hydrogen, C₁₋₆-alkyl, or form a ring which may contain a further hetero atom,
   whereby if D is N, then E, F and G may not simultaneously be C—R⁴, C—R⁵ or C—R⁶ or D, E, F and G may not simultaneously be C—R³, C—R⁴, C—R⁵ or C—R⁶, as well as the isomers and salts thereof.

The compounds of the general formula I which have proved to be especially valuable are those in which
A is the group═NR⁷,
W is oxygen,
Z is a bond,
R¹ is branched or unbranched C₁₋₁₂-alkyl or C₂₋₁₂-alkenyl which is optionally substituted once or many times by halogen, hydroxy, C₁₋₆-alkyloxy, aralkyloxy, C₁₋₆-alkyl and/or NR¹¹R¹² or C₃₋₁₀-cycloalkyl or C₃₋₁₀-cycloalkenyl which is optionally substituted once or many times by halogen, hydroxy, C₁₋₆-alkyloxy, C₁₋₆-alkyl and/or NR¹¹R¹²; or aryl or hetaryl which is optionally substituted once or many times by halogen, hydroxy, C₁₋₆-alkyloxy, aralkyloxy, C₁₋₆-alkyl and/or by C₁₋₆-alkyl which is substituted once or many times by halogen,
X is C₁₋₆-alkyl;
R² signifies monocyclic aryl, bicyclic aryl or heteroaryl, which is unsubstituted or optionally substituted once or many times by halogen, C₁₋₆-alkyl, C₁₋₆-alkoxy and/or hydroxy and
D signifies N or C—R³,
E signifies N or C—R⁴,
F signifies N or C—R⁵, and
G signifies N or C—R⁶, whereby
R³, R⁴, R⁵ and R⁶ are hydrogen, halogen; or C₁₋₆alkoxy, C₁₋₆-alkyl, C₁₋₆-carboxyalkyl either unsubstituted or optionally substituted once or many times by halogen,
R⁷ is hydrogen or C₁₋₆-alkyl,
R⁹ is hydrogen or C₁₋₆-alkyl and
R¹¹ and R¹² are hydrogen, C₁₋₆-alkyl, or form a ring which may contain a further hetero atom,
   whereby if D is N, then E, F and G may not simultaneously be C—R⁴, C—R⁵ or C—R⁶ or D, E, F and G may not simultaneously be C—R³, C—R⁴, C—R⁵ or C—R⁶, as well as the isomers and salts thereof.

The compounds of the general formula I which are particularly effective are those, in which
A is the group═NR⁷,
W is oxygen,
Z is a bond,
R¹ is phenyl, quinolyl, isoquinolyl, indazolyl or C₅₋₆-cycloalkyl, which, independently of one another, are optionally substituted once or many times by halogen, trifluoromethyl, methoxy and/or C₁₋₄-alkyl,
X is C₁₋₆-alkyl;
R² is pyridyl and
D signifies N or C—R³,
E signifies N or C—R⁴,
F signifies N or C—R⁵ and
G signifies N or C—R⁶, whereby
R³, R⁴, R⁵ and R⁶ are hydrogen, and
R⁷ and R⁹ are hydrogen,
   whereby if D is N, then E, F and G may not simultaneously be C—R⁴, C—R⁵ or C—R⁶ or D, E, F and G may not simultaneously be C—R³, C—R⁴, C—R⁵ or C—R⁶, as well as the isomers and salts thereof.

The compounds according to the invention prevent phosphorylation, i.e. certain tyrosine kinases can be selectively inhibited, whereby the persistent angiogenesis can be stopped. In this way, for example, the growth and spread of tumours is suppressed.

The compounds of the general formula I according to the invention also contain the possible tautomeric forms and include the E- or Z-isomers, or if a chiral centre is present, also the racemates and enantiomers.

Owing to their inhibitory activity in respect of phosphorylation of the VEGF receptor, the compounds of formula I and their physiologically acceptable salts may be used as medicaments. Owing to their profile of activity, the compounds according to the invention are suitable for treating diseases caused by or accelerated by persistent angiogenesis.

Since the compounds of formula I are identified as inhibitors of KDR and FLT tyrosine kinase, they are especially suitable for treating those diseases that are caused by or accelerated by the persistent angiogenesis, triggered by the VEGF receptor, or by an increase in vascular permeability.

The object of the present invention is also the use of the compounds according to the invention as inhibitors of KDR and FLT tyrosine kinase.

A further object of the present invention is thus the medicaments for treating tumours, and their use.

The compounds according to the invention may be used either on their own or in a formulation as a medicament for treating psoriasis, arthritis, such as rheumatoid arthritis, haemangioma, angiofibroma, eye diseases such as diabetic retinopathy, neovascular glaucoma, kidney diseases such as glomerulonephritis, diabetic nephropathy, malignant nephrosclerosis, thrombic microangiopathic syndrome, transplantation rejections and glomerulopathy, fibrotic diseases such as cirrhosis of the liver, mesangial cell proliferation diseases, artheriosclerosis and injuries to nerve tissue.

When treating injuries to nerve tissue, the compounds according to the invention can prevent rapid formation of scar tissue at the site of the wounds, i.e. the onset of scar formation is prevented before the axons join together again. Thus, reconstruction of the nerve unions is simplified.

In addition, the compounds according to the invention suppress ascites formation in patients. Similarly, VEGF-induced oedema are suppressed. Such medicaments, their formulations and uses are likewise objects of the present invention.

The invention further relates to the use of the compounds of the general formula I in the production of a medicament for treating tumours, psoriasis, arthritis, such as rheumatoid arthritis, haemangioma, angiofibroma, eye diseases such as diabetic retinopathy, neovascular glaucoma, kidney diseases such as glomerulonephritis, diabetic nephropathy, malignant nephrosclerosis, thrombic microangiopathic syndrome, transplantation rejections and glomerulopathy, fibrotic diseases such as cirrhosis of the liver, mesangial cell proliferation diseases, artheriosclerosis and injuries to nerve tissue.

When using the compounds of formula I as medicaments, they are brought into the form of a pharmaceutical preparation, which contains, in addition to the active ingredient for enteral or parenteral application, appropriate pharmaceutical, organic or inorganic inert carriers, for example water, gelatin, gum arabic, lactose, starch, magnesium stearate, talc, vegetable oils, polyalkylene glycols etc. The pharmaceutical preparations may exist in solid form, for example as tablets, coated tablets, suppositories, capsules, or in liquid form, for example as solutions, suspensions or emulsions. They may additionally contain excipients such as preservatives, stabilizers, wetting agents or emulsifiers, salts to change the osmotic pressure or buffers.

For parenteral application, injection solutions or suspensions are especially suitable, particularly aqueous solutions of the active compounds in polyhydroxy-ethoxylated castor oil.

Surface-active excipients may also be used as carrier systems, for example salts of bile acid or animal or vegetable phospholipids, and also mixtures thereof, as well as liposomes or constituents thereof.

For oral application, tablets, coated tablets or capsules are especially suitable, with talcum and/or hydrocarbon carriers or binders, for example lactose, corn starch or potato starch. Application may also be carried out in liquid form, for example as juice, to which a sweetener may optionally be added, and if necessary a flavouring agent.

Dosaging of the active ingredients may vary according to the mode of administration, the age and the weight of the patient, the nature and severity of the illness to be treated and similar factors. The daily dose is 0.5-1000 mg, preferably 50-200 mg, whereby the dose may be given as a single dose to be administered once or may be divided into two or more daily doses. The above-described formulations and dosage forms are likewise objects of the present invention.

Preparation of the compounds according to the invention is carried out according to known methods. For example, the compounds of formula I are obtained, whereby
a) in a compound of formula II, 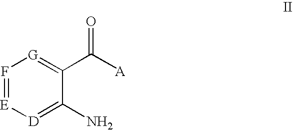
   wherein D to G are defined as above, and A is OR¹³, whereby OR¹³ is hydrogen or C₁₋₄-alkyl or C₁₋₄-acyl, first of all the amine is alkylated and then COA is converted into an amide, or NH2 is converted into halogen, A is converted into an amide and halogen is converted into the corresponding amine,
   or
b) in a compound of formula III, 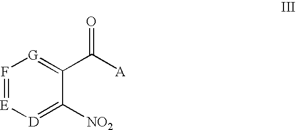
   wherein D to G are defined as above, and A is halogen or OR¹³, whereby R¹³ may be hydrogen, lower alkyl or acyl, COA is converted into an amide, the nitro group is reduced to the amine and is then alkylated,
   or
c) in a compound of formula IV, 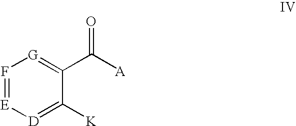
   wherein D to G are defined as above and K is hydroxy or halogen, and A is halogen or OR¹³, whereby OR¹³ may be hydrogen, lower alkyl or acyl, K is converted into an amine, COA is converted into an amide, or if K is hydroxy it is converted into halogen, and the procedure continues as above.

In all cases, the sequence of steps can be switched.

Amide formation is effected according to methods known in literature. An amide may be formed from a corresponding ester. The ester is reacted in accordance with J. Org. Chem. 1995, 8414 with aluminium trimethyl and the corresponding amine in solvents such as toluene at temperatures of 0° C. to boiling point of the solvent. If the molecule contains two ester groups, both are converted to the same amide.

When using nitriles instead of the ester, amidines are obtained under analogous conditions.

To form the amide, all methods that are known from peptide chemistry may be used. For example, the corresponding acid can be reacted with the amine in aprotic polar solvents, for example dimethylformamide via an activated acid derivative, for example obtainable with hydroxybenzotriazole and a carbodiimide, such as diisopropyl carbodiimide, or also with prepared reagents, for example HATU (Chem. Comm. 1994, 201, or BTU, at tempertures between 0° C. and boiling point of the solvent. To form the amide, the method using the mixed acid anhydride, the acid chloride, the imidazolide or the azide may also be employed. For reactions of the acid chloride, the solvent dimethyl acetamide is preferred at temperatures from room temperature to boiling point of the solvent, preferably 80-100° C.

If different amide groups are to be introduced into the molecule, the second ester group must be introduced into the molecule for example after producing the first amide group, and is then amidated, or a molecule exists in which one group is present as an ester and the other as an acid, and the two groups are amidated after each other by different methods.

Thioamides may be obtained from the anthranilamides by a reaction with diphosphadithianes according to Bull Soc.Chim.Belg. 87, 229, 1978 or by a reaction with phosphorus pentasulfide in solvents such as pyridine or also without any solvents at all at temperatures of 0° C. to 200° C.

Reduction of the nitro group is carried out in polar solvents at room temperature or at elevated temperature. Suitable catalysts for reduction are metals such as Raney nickel or noble metal catalysts such as palladium or platinum or also palladium hydroxide, optionally on carriers. Instead of hydrogen, ammonium formate, cyclohexene or hydrazine may also be used, for example, in known manner. Reducing agents such as tin II chloride or titanium (III) chloride may be used in the same way as complex metal hydrides, optionally in the presence of heavy metal salts. Iron may also be used as a reducing agent. In this case, the reaction is carried out in the presence of an acid, such as acetic acid or ammonium chloride, optionally adding a solvent, for example water, methanol, iron/ammonia etc. In the prolonged reaction time in this variant, acylation of the amino group can occur.

If alkylation of an amino group is desired, alkylation may be effected by the usual methods—for example with alkyl halides—or by the Mitsonubo variant by a reaction with an alcohol in the presence of for example triphenylphosphine and azodicarboxylic acid ester. The amine may also undergo reductive alkylation with aldehydes or ketones, whereby the reaction is carried out in the presence of a reducing agent, for example sodium cyanoborohydride in a suitable inert solvent, for example ethanol, at temperatures from 0° C. to boiling point of the solvent. When starting with a primary amino group, the reaction is effected optionally with two different carbonyl compounds after one another, whereby mixed derivatives are obtained [literature e.g. Verardo et al. Synthesis (1993), 121; Synthesis (1991), 447; Kawaguchi, Synthesis (1985), 701; Micovic et al. Synthesis (1991), 1043].

It may also be advantageous to firstly form the Schiff's base by reacting the aldehyde with the amine in solvents such as ethanol or methanol, optionally adding excipients such as glacial acetic acid, and only then to add reducing agents, e.g. sodium cyanoborohydride.

Ether cleavages are carried out by conventional methods known from literature. Here, even if several groups are present in the molecule, selective cleavage can be achieved. The ether is treated for example with boron tribromide in solvents such as dichloromethane at temperatures between −100° C. and boiling point of the solvent, preferably at −78° C. However, it is also possible to cleave the ether by means of sodium thiomethylate in solvents such as dimethylformamide. The temperature may be between room temperature and boiling point of the solvent, preferably 150° C. In the case of benzyl ethers, cleavage is also effected with strong acids, for example trifluoroacetic acid, at temperatures from room temperature to boiling point.

The transformation of a hydroxy group, which is in ortho- or para-position to a nitrogen of a 6-ring hetaryl, into halogen, may be carried out for example by a reaction with inorganic acid halides, for example phosphorus oxychloride, optionally in a solvent, at temperatures of up to boiling point of the solvent or of the acid halide.

Substitution of a halogen, tosylate, triflate or nonaflate, which are in ortho- or para-position to a nitrogen in a 6-membered heteroaromatic, takes place by a reaction with a corresponding amine in inert solvents, for example xylene, or in polar solvents, such as N-methylpyrrolidone or dimethylacetamide, at temperatures of 60-170° C. It is however also possible to effect heating without solvents. The addition of an auxiliary base such as potassium carbonate or cesium carbonate or the addition of copper and/or copper oxide may be advantageous. A palladium-catalysed reaction is also possible.

Introduction of the halogens chlorine, bromine or iodine via an amino group may also take place for example according to Sandmeyer, by reacting the diazonium salts formed with nitrites as an intermediate, with copper (I) chloride or copper (I) bromide in the presence of the corresponding acid such as hydrochloric acid or hydrobromic acid or with potassium iodide.

If an organic nitrous acid ester is used, the halogens may be introduced e.g. by adding methylene iodide or tetrabromomethane in a solvent such as dimethyl-formamide. Removal of the amino group may be accomplished either by a reaction with an organic nitrous acid ester in tetrahydrofuran or by diazotising the diazonium salt and boiling it down reductively for example with phosphorus acid, optionally adding copper (I) oxide.

Fluorine is introduced for example by the Balz-Schiemann reaction of diazonium tetrafluoroborate or according to J. Fluor. Chem. 76, 1996, 59-62 by diazotizing in the presence of HFxpyridine with subsequent boiling down optionally in the presence of a source of fluoride ions, e.g. tetrabutylammonium fluoride.

The isomeric mixtures can be separated by conventional methods, for example crystallisation, any form of chromatography or by salt formation, into the enantiomers or E/Z-isomers.

Production of the salts takes place in conventional manner, by mixing a solution of the compound of formula I with the equivalent amount or with an excess of a base or acid, which is optionally in solution, and separating the precipitate or by working up the solution in conventional manner.

Insofar as the production of the intermediates is not described, these are known or may be produced analogously to known compounds or analogously to the processes described here.

The intermediates described are especially suitable for the production of the aza- and polyazanthranylamides according to the invention.

These intermediates are likewise an object of the present invention.

The intermediates are partly self-active and may therefore similarly be used in the production of a medicament for treating tumours, psoriasis, arthritis, such as rheumatoid arthritis, haemangioma, angiofibroma, eye diseases such as diabetic retinopathy, neovascular glaucoma, kidney diseases such as glomerulonephritis, diabetic nephropathy, malignant nephrosclerosis, thrombic microangiopathic syndrome, transplantation rejections and glomerulopathy, fibrotic diseases such as cirrhosis of the liver, mesangial cell proliferation diseases, artheriosclerosis and injuries to nerve tissue.

The following examples illustrate the preparation of the compounds according to the invention without limiting the scope of the compounds being claimed to these examples.

Preparation of the Intermediates

The following examples illustrate the preparation of the intermediates according to the invention which are especially suitable for producing the compounds of general formula I according to the invention, without limiting the invention to these examples.

A. 3-aminopyridine-2-carboxylic acid methyl ester

4 g (29 mmols) of 3-aminopyridine-2-carboxylic acid are placed in a mixture of 58 ml of methanol and 200 ml of toluene under argon, whilst excluding moisture, and then mixed dropwise at room temperature with 21.7 ml (43.4 mmols) of a 2M solution of trimethylsilyl diazomethane in hexane. After stirring for 2 h at room temperature, the mixture is concentrated in a vacuum, the residue taken up in 100 ml of 1N sodium hydroxide solution, and extracted three times, each time with 100 ml of ethyl acetate. The organic phase collected is washed with water, dried, filtered and concentrated. 2.27 g of 3-aminopyridine-2-carboxylic acid methyl ester are obtained.

B. N-isoquinolin-3-yl(3-aminopyridine)-2-carboxylic acid amide

215 mg (1.4 mmols) of 3-aminopyridine-2-carboxylic acid methyl ester are placed in 15 ml of toluene under argon, whilst excluding moisture, and then mixed in succession with 224 mg (1.55 mmols) of 3-aminoisoquinoline and 0.78 ml of trimethyl aluminium solution (2.5 M in toluene). Stirring is subsequently effected for 2 h at a bath temperature of 120° C. After cooling, the mixture is mixed with 30 ml of a saturated sodium carbonate solution, and extracted three times, each time with 30 ml of ethyl acetate. The ethyl acetate phase is washed with water, dried, filtered and concentrated. The residue is stirred with ethyl acetate/hexane. 211 mg (56% of theory) of N-isoquinolin-3-yl(3-aminopyridine)-2-carboxylic acid amide are obtained.

C. 4-[(4-pyridyl)methyl]amino-pyrimidine-5-carboxylic acid methyl ester

5.85 g of commercial 4-hydroxypyrimidine-5-carboxylic acid methyl ester are mixed with 5.3 ml of triethylamine and 38 ml of phosphorus oxychloride and stirred for 3 hours at 140° C. The mixture is concentrated by evaporation to dryness and mixed with 100 ml of toluene. 19.2 ml of 4-aminomethylpyridine are added dropwise at room temperature and the mixture is subsequently stirred for 1 hour at 130° C. The solid is filtered by suction and washed three times, each time with 250 ml of toluene. The filtrate is concentrated, purified by column chromatography and recrystallised from ethanol. 4.9 g (53% of theory) of 4-[(4-pyridyl)methyl]amino-pyrimidine-5-carboxylic acid methyl ester are obtained. m.p.: 111-112° C.

D. 3-[(4-pyridyl)methyl]amino-pyrazine-2-carboxylic acid methyl ester

1. 4.0 g of 3-aminopyrazine-2-carboxylic acid methyl ester are dissolved in a mixture of 26 ml of concentrated hydrochloric acid and 26 ml of water. At <5° C. a solution of 1.99 g of sodium nitrite in 21.5 ml of water is added dropwise. This solution is slowly added dropwise to 43 ml of a saturated solution of sodium chloride in water. After 15 minutes, the solution is neutralised with solid sodium hydrogen carbonate, diluted with water and extracted with methylene chloride. The organic extracts are dried over sodium carbonate and concentrated. After column chromatography (hexane/ethyl acetate), 1.71 g (38% of theory) of 3-chloropyrazine-2-carboxylic acid methyl ester are obtained. m.p.: 30° C.
2. 0.80 g of 3-chloro-pyrazine-2-carboxylic acid methyl ester are dissolved in 10 ml of 2-propanol and mixed with 0.47 ml of 4-aminomethylpyridine. The mixture is heated at reflux for 24 hours. The solvent is subsequently distilled off and the residue purified by column chromatography (methylene chloride/methanol). 975 mg (44% of theory) of 3-[(4-pyridyl)methyl]amino-pyrazine-2-carboxylic acid methyl ester are obtained. M.p.: 95° C.
   E. 3-pyridylmethylaminopyridine-2-carboxylic acid methyl ester

302 mg (2.2 mmols) of 3-aminopyridine-2-carboxylic acid methyl ester in 13 ml of methanol are mixed with 0.05 ml of glacial acetic acid and 374 mg (3.5 mmols) of 4-pyridine carbaldehyde and stirred for 24 h at room temperature. Then, whilst cooling in an ice bath, 228 mg (3.6 mmols) of sodium cyanoborohydride are added and stirred for 24 h at room temperature. The preparation is rotated, the residue taken up in 25 ml of water and extracted three times, each time with 25 ml of ethyl acetate. The organic phase is dried, filtered and concentrated. The residue is chromatographed over silica gel with methylene chloride:ethanol=10:1 as eluant. After combining the corresponding fractions, 130 mg (17% of theory) of 3-pyridylmethylaminopyridine-2-carboxylic acid methyl ester are obtained.

F. 3-pyridylmethylaminopyridine-2-carboxylic acid

3 g (12.4 mmols) of 3-pyridylmethylaminopyridine-2-carboxylic acid methyl ester in 50 ml of ethanol are mixed with 15 ml of 1N sodium hydroxide solution and heated for 2 h to a bath temperature of 100° C. After distilling off the ethanol, the mixture is diluted with water and extracted once with ethyl acetate. It is then neutralised with 3N hydrochloric acid, and the precipitated product is filtered by suction. 1.5 g of 3-pyridylmethylaminopyridine-2-carboxylic acid are obtained.

The following examples describe the preparation of the compounds according to the invention without limiting it to these examples.

### Example 1.0

### Preparation of N-isoguinolin-3-yl-3-[(4-pyridyl)methyl]amino-pyridine-2-carboxylic acid amide

190 mg (0.72 mmols) of N-isoquinolin-3-yl(3-aminopyridine)-2-carboxylic acid amide in 13 ml of methanol are mixed with 0.05 ml of glacial acetic acid and 123 mg (1.15 mmols) of 4-pyridine carbaldehyde and stirred for 24 h at room temperature. Then, whilst cooling in an ice bath, 72mg (1.15 mmols) of sodium cyanoborohydride are added and stirred for 24 h at room temperature. The preparation is rotated, the residue taken up in 25 ml of water and extracted 15 three times, each time with 25 ml of ethyl acetate. The organic phase is dried, filtered and concentrated. The residue is chromatographed over silica gel with methylene chloride:ethanol=95:5 as eluant. After combining the corresponding fractions, 43 mg (17% of theory) of N-isoquinolin-3-yl-3-[(4-pyridyl)-methyl]amino-pyridine-2-carboxylic acid amide are obtained with a melting point of 167.9° C.

The following are produced in analogous manner:

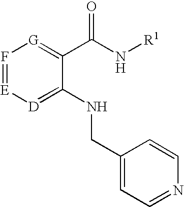

| | | | | | | melting |
|---|---|---|---|---|---|---|
| Exam- | | | | | | point |
| ple | R¹ | D | E | F | G | ° C. |
| 1.1 | 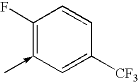 | CH | CH | CH | N | |
| 1.2 | 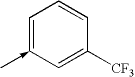 | CH | CH | CH | N | |
| 1.3 | 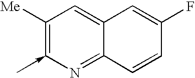 | CH | CH | CH | N | |
| 1.4 | 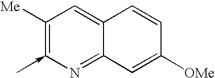 | CH | CH | CH | N | |
| 1.5 | 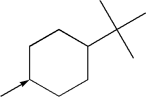 | CH | CH | CH | N | |
| 1.6 | 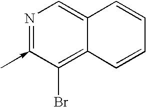 | CH | CH | CH | N | |
| 1.7 | 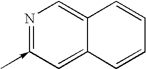 | CH | N | CH | CH | |
| 1.8 | 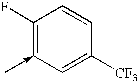 | CH | N | CH | CH | |
| 1.9 | 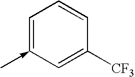 | CH | N | CH | CH | |
| 2.0 | 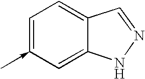 | CH | N | CH | CH | |

### Example 2.0

### Preparation of N-isoguinolin-3-yl-4-[(4-pyridyl)methyl]amino-pyrimidine-5-carboxylic acid amide

216 mg of 3-aminoisoquinoline are placed in 10 ml of toluene under nitrogen, whilst cooling with ice. 0.65 ml of a 2 molar solution of trimethyl aluminium in toluene are added dropwise and the mixture stirred for 10 minutes. Then, 318 mg of 4-[(4-pyridyl)methyl]amino-pyrimidine-5-carboxylic acid methyl ester are added and the mixture heated at 120° C. for 3.5 hours. After adding 0.2 ml of a 2 molar solution of trimethyl aluminium in toluene, heating continues for 7 hours at 120° C. After cooling, the reaction mixture is added to a solution of 144 mg of 3-aminoisoquinoline in 0.65 ml of a 2 molar solution of trimethyl aluminium in toluene and again heated at 120° C. for 7 hours. The solvent is subsequently distilled off and the residue mixed with sodium hydrogen carbonate solution. The mixture is extracted with ethyl acetate several times. The extract is purified by column chromatography and recrystallised from ethanol. 129 mg (24% of theory) of N-isoquinolin-3-yl-4-[(4-pyridyl)-methyl]amino-pyrimidine-5-carboxylic acid amide are obtained. M.p.: 218-220° C.

The following are produced in analogous manner:

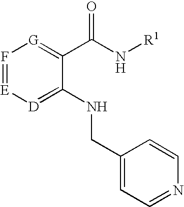

| Ex- | | | | | | melting |
|---|---|---|---|---|---|---|
| am- | | | | | | point |
| ple | R¹ | D | E | F | G | ° C. |
| 2.1 | 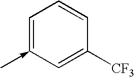 | N | CH | N | CH | 199-200 |
| 2.2 | 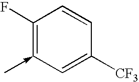 | N | CH | N | CH | |
| 2.3 | 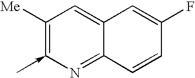 | N | CH | N | CH | |
| 2.4 | 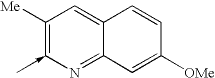 | N | CH | N | CH | |
| 2.5 | 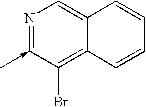 | N | CH | N | CH | |
| 2.6 | 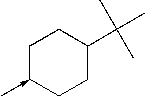 | N | CH | N | CH | |
| 2.7 | 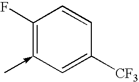 | CH | CH | N | CH | |
| 2.8 | 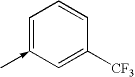 | CH | CH | N | CH | |
| 2.9 | 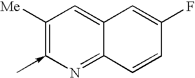 | CH | CH | N | CH | |
| 2.10 | 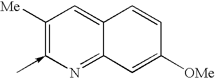 | CH | CH | N | CH | |
| 2.11 | 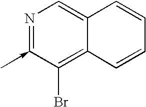 | CH | CH | N | CH | |
| 2.12 | 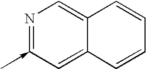 | CH | CH | N | CH | |

### Example 3.0

### Preparation of N-isoguinolin-3-yl-3-[(4-pyridyl)methyl]amino-pyrazine-2-carboxylic acid amide

288 mg of 3-aminoisoquinoline are placed in 10 ml of toluene under nitrogen, whilst cooling with ice. 1 ml of a 2 molar solution of trimethyl aluminium in toluene is added dropwise and the mixture stirred for 10 minutes. Then, 244 mg of 3-[(4-pyridyl)methyl]amino-pyrazine-2-carboxylic acid methyl ester are added and the mixture heated at 120° C. for 4 hours. After cooling, the mixture is diluted with ethyl acetate, washed with saturated sodium chloride solution, dried over sodium sulphate and filtered by suction through Celite. The filtrate is concentrated and purified by column chromatography (hexane/ethyl acetate). 150 mg (42% of theory) of N-isoquinolin-3-yl-3-[(4-pyridyl)methyl]amino-pyrazine-2-carboxylic acid amide are obtained. M.p.: 139° C.

### Example 4.0

### Preparation of N-indazol-5-yl-3-[(4-pyridyl)methyl]amino-pyridine-2-carboxylic acid amide

229 mg (1 mmol) of 3-pyridylmethylaminopyridine-2-carboxylic acid in 10 ml of dimethylformamide are stirred with 280 mg (1 mmol) of 5-aminoindazole, 253 mg (2.5 mmols) of N-methylmorpholine and 456 mg (1.2 mmols) of O—(7-azabenzotriazol-1-yl)-1,1,3,3-tetramethyluronium hexafluorophosphate (HATU) at room temperature for 3 hours under argon, whilst excluding moisture. Then, the mixture is diluted with diluted sodium hydrogen carbonate solution and extracted with ethyl acetate. The ethyl acetate phase is dried, filtered and concentrated. After chromatography on silica gel with methylene chloride:ethanol=10:1 as elulant, 100 mg (27% of theory) of N-indazol-5-yl-3-[(4-pyridyl)methyl]amino-pyridine-2-carboxylic acid amide are obtained.

Solutions Required for the Tests

Stock solutions
Stock solution A: 3mM ATP in water pH 7.0 (−70° C.)
Stock solution B: g-33P-ATP 1mCi/100 μl
Stock solution C: poly-(Glu4Tyr) 10 mg/ml in water
   Solution for dilutions
Substrate solvent: 10 mM DTT, 10 mM manganese chloride 100 mM magnesium chloride
Enzyme solution: 120 mM Tris/HCI, pH 7, 5, 10 μM sodium vanadium oxide

The following application examples illustrate the biological activity and use of the compounds according to the invention without limiting them to the examples.

### Application Example 1

Inhibition of KDR- and FLT-1 kinase activity in the presence of the compounds according to the invention

10 μl of substrate mix [10 μl vol ATP stock solution A+25 μCi g-33P-ATP (ca. 2.5 μl of stock solution B)+30 μl poly-(Glu4Tyr) stock solution C+1.21 ml substrate solvent], 10 μl inhibitor solution [substances corresponding to the dilutions, as a control 3% DMSO in substrate solvent] and 10 μl enzyme solution [11.25 μg enzyme stock solution (KDR or FLT-1 kinase) are diluted at 4° C. in 1.25 ml enzyme solution], are added to a tapering microtitre plate (without protein binding). The mixture is mixed thoroughly and incubated for 10 minutes at room temperature. Subsequently, 10 μl of stop solution (250 mM EDTA, pH 7.0) is added, mixed and 10 μl of the solution transferred to a P 81 phosphocellulose filter. Is is subsequently washed several times in 0.1 M phosphoric acid. The filter paper is dried, coated with MeltiLex and measured in a MicroBeta counter.

The IC50 values are determined from the concentration of inhibitor required to inhibit the phosphate incorporation to 50% of the uninhibited incorporation after deducting the reference value (EDTA-stopped reaction).

The results of the kinase inhibition IC50 in μM are illustrated in the following table.

| | VEGFR II |
|---|---|
| Example No. | (KDR, nM) |
| 2.0 | 100 |
| 2.1 | 200 |

1-12. (canceled)

13. A compound of the general formula 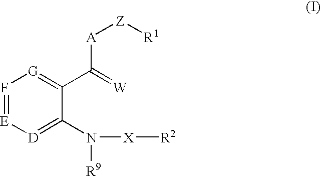
in which
A is the group NR⁷,
W is oxygen, sulfur, two hydrogen atoms or the group —NR⁸;
Z is a bond, the group NR¹⁰ or ═N—, branched or unbranched C₁₋₁₂alkyl or the group 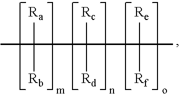
whereby
m, n and o are 0-3;
Rₐ, R_{b}, R_{c}, R_{d}, Rₑ, R_{f}, independently of one another, are hydrogen, fluorine, C₁₋₄alkyl or the group —NR¹⁰ and/or Rₐ and/or R_{b} with R_{c} and/or R_{d} or R_{c} with Rₑ and/or R_{f} may form a bond, or up to two of radicals Rₐ-R_{f} may close a bridge to R¹ or to R⁷ each with up to three carbon atoms, is branched or unbranched C₁₋₁₂alkyl or C₂₋₁₂alkenyl which is optionally substituted once or many times by halogen, hydroxy, C₁₋₆alkyloxy, aralkyloxy, C₁₋₆alkyl and/or NR¹¹R¹²; or C₃₋₁₀cycloalkyl or C₃₋₁₀cycloalkenyl which is optionally substituted once or many times by halogen;
X is C¹⁻⁶alkyl;
R¹ is branched or unbranched C₁₋₁₂alkyl or C₂₋₁₂alkenyl which is optionally substituted once or many times by halogen, hydroxy, C₁₋₆alkyloxy, aralkyloxy, C₁₋₆alkyl and/or NR¹¹R¹²; or C₃₋₁₀cycloalkyl or C₃₋₁₀cycloalkenyl which is optionally substituted once or many times by halogen, hydroxy, C₁₋₆alkyloxy, C₁₋₆alkyl and/or NR¹¹R¹²; or aryl or hetaryl which is optionally substituted once or many times by halogen, hydroxy, C₁₋₆alkyloxy, aralkyloxy, C₁₋₆alkyl and/or by C₁₋₆alkyl which is substituted once or many times by halogen;
R² signifies monocyclic aryl, bicyclic aryl or heteroaryl, which is unsubstituted or optionally substituted once or many times by halogen, C₁₋₆alkyl, C₁₋₆alkoxy and/or hydroxy and
D signifies C—R³,
E signifies N or C—R⁴,
F signifies N or C—R⁵ and
G signifies N or C—R⁶, provided that one of E, F and G are N;
whereby R³, R⁴, R⁵ and R⁶ are hydrogen, halogen; or C₁₋₆alkoxy, C₁₋₆alkyl, C₁₋₆carboxyalkyl either unsubstituted or optionally substituted once or many times by halogen;
R⁷ is hydrogen or C₁₋₆alkyl or with Rₐ-R_{f} forms a bridge of Z or to R¹ with up to 3 ring members;
R⁸, R⁹ and R¹⁰ are hydrogen or C₁₋₆alkyl; and
R¹¹ and R¹² are hydrogen, C₁₋₆alkyl, or form a ring which may contain a further hetero atom and salts thereof.

14. A compound of general formula (I), according to claim 13, in which
A is the group NR7;
W is oxygen, sulfur, two hydrogen atoms or the group NR⁸;
Z is a bond;
R¹ is branched or unbranched C₁₋₁₂alkyl or C₂₋₁₂alkenyl which is optionally substituted once or many times by halogen, hydroxy, C₁₋₆alkyloxy, aralkyloxy, C₁₋₆alkyl and/or NR¹¹R¹²; or C₃₋₁₀cycloalkyl or C₃₋₁₀cycloalkenyl which is optionally substituted once or many times by halogen, hydroxy, C₁₋₆alkyloxy, C₁₋₆alkyl and/or NR¹¹R¹²; or aryl or hetaryl which is optionally substituted once or many times by halogen, hydroxy, C₁₋₆alkyloxy, aralkyloxy, C₁₋₆alkyl and/or by C₁₋₆alkyl which is substituted once or many times by halogen;
X is C₁₋₆alkyl;
R² signifies monocyclic aryl, bicyclic aryl or heteroaryl, which is unsubstituted or optionally substituted once or many times by halogen, C₁₋₆alkyl, C₁₋₆alkoxy and/or hydroxy and
D signifies C—R³,
E signifies N or C—R⁴,
F signifies N or C—R⁵ and
G signifies N or C—R⁶, provided that one of E, F and G are N;
whereby R³, R⁴, R⁵ and R⁶ are hydrogen, halogen; or C₁₋₆alkoxy, C₁₋₆alkyl, C₁₋₆carboxyalkyl either unsubstituted or optionally substituted once or many times by halogen;
R⁷ is hydrogen or C₁₋₆alkyl;
R⁸ and R⁹ are hydrogen or C₁₋₆alkyl; and
R¹¹ and R¹² are hydrogen, C₁₋₆alkyl, or form a ring which may contain a further hetero atom or salts thereof.

15. a compound of general formula (I), according to claim 13, in which
A is the group NR⁷;
W is oxygen;
Z is a bond;
R¹ is branched or unbranched C₁₋₁₂alkyl or C₂₋₁₂alkenyl which is optionally substituted, independently of each another, once or many times by halogen, hydroxy, C₁₋₆alkyloxy, aralkyloxy, C₁₋₆alkyl and/or NR¹¹R¹²; or C₃₋₁₀cycloalkyl or C₃₋₁₀cycloalkenyl which is optionally substituted once or many times by halogen, hydroxy, C₁₋₆alkyloxy, C₁₋₆alkyl and/or NR¹¹R¹²; or aryl or hetaryl which is optionally substituted once or many times by halogen, hydroxy, C₁₋₆alkyloxy, aralkyloxy, C₁₋₆alkyl and/or by C₁₋₆alkyl which is substituted once or many times by halogen,
X is C₁₋₆alkyl;
R² signifies monocyclic aryl, bicyclic aryl or heteroaryl, which is unsubstituted or optionally substituted once or many times by halogen, C₁₋₆alkyl, C₁₋₆alkoxy and/or hydroxy and
D signifies C—R³,
E signifies N or C—R⁴,
F signifies N or C—R⁵ and
G signifies N or C—R⁶, provided that one of E, F and G are N;
whereby R³, R⁴, R⁵ and R⁶ are hydrogen, halogen; or C₁₋₆alkoxy, C₁₋₆alkyl, C₁₋₆carboxyalkyl either unsubstituted or optionally substituted once or many times by halogen;
R⁷ is hydrogen or C₁₋₆alkyl;
R⁹ is hydrogen or C₁₋₆alkyl; and
R¹¹ and R¹² are hydrogen, C₁₋₆alkyl, or form a ring which may contain a further hetero atom or salts thereof.

16. A compound of general formula I, according to claim 13, in which
A is the group NR⁷;
W is oxygen;
Z is a bond;
R¹ is phenyl, quinolyl, isoquinolyl, indazolyl or C₅₋₆cycloalkyl, which, independently of one another, are optionally substituted once or many times by halogen, trifluoromethyl, methoxy and/or C₁₋₄alkyl;
X is C₁₋₆alkyl;
R² is pyridyl and
D signifies C—R³,
E signifies N or C—R⁴,
F signifies N or C—R⁵ and
G signifies N or C—R⁶, provided that one of E, F and G. are N;
whereby R³, R⁴, R⁵ and R⁶ are hydrogen; and
R⁷ and R⁹ are hydrogen or salts thereof.

17. A pharmaceutical composition comprising one or more compound according to claim 13 and a pharmaceutically acceptable carrier.

18. A method for treating breast cancer by inhibiting an enzyme selected from KDR and FLT tyrosine kinase, which comprises contacting the enzyme with a compound of claim 13.

19. A pharmaceutical composition of claim 18 for enteral, parenteral or oral application.

20. A compound selected from the group consisting of
A. 3-aminopyridine-2-carboxylic acid methylester;
B. N-isoquinolin-3-yl(3-aminopyridine)-2-carboxylic acid amide;
C. 4-[(4-pyridyl)methyl]amino-pyrimidine-5-carboxylic acid methyl ester;
D. 3-[(4-pyridyl)methyl]amino-pyrazine-2-carboxylic acid methyl ester;
E. 3-pyridylmethylaminopyridine-2-carboxylic acid methyl ester; and
F. 3-pyridylmethylaminopyridine-2-carboxylic acid.
